(19) United States
(12) Patent Application Publication
(10) Pub. No.: US 2006/0134774 A1
(43) Pub. Date: Jun. 22, 2006

(54) Detection of protease enzymes

(75) Inventors: Peter A Clausen, Ijamsville, MD (US); James G Lazar, Bethasda, MD (US)

Correspondence Address: HELLER EHRMAN WHITE & MCAULIFFE LLP, 1717 RHODE ISLAND AVE, NW, WASHINGTON, DC, 20036-3001, US

(73) Assignee: Marligen Biosciences, Inc., Ijamsville, MD (US)

(21) Appl. No.: 10/535,833

(22) PCT Filed: Nov. 24, 2003
(86) PCT No.: PCT/US03/37514

§ 371 Date: Oct. 24, 2005

Related U.S. Application Data
(60) Provisional application No. 60/428,286, filed on Nov. 22, 2002.

(51) Int. Cl. C12M 1/34; G01N 33/543; G01N 33/551

(52) U.S. Cl. 435/287.2; 436/518; 436/524

(57) ABSTRACT

Methods and compositions for the detection of biomolecules, for example, proteases, are provided. The novel compositions, methods, and kits of the present invention have broad applicability in the detection of proteases, and providing enhanced specificity in the detection of proteases. The compositions and methods may be used to measure the activities of multiple proteases simultaneously or in a multiplexed format, particularly in planar and liquid array formats.

This application claims priority to U.S. Provisional Application Ser. No. 60/428,286, filed Nov. 22, 2002, the contents of which are hereby incorporated by reference in their entirety.

### FIELD OF THE INVENTION

The present invention provides methods and compositions for the detection of biomolecules. In particular, the present invention relates to compositions and methods for measuring the activities of proteases. The novel compositions, methods, and kits of the present invention have broad applicability in the detection of proteases, and providing enhanced specificity in the detection of proteases. Furthermore, the compositions and methods of the present invention will find broad use in measuring the activities of multiple proteases simultaneously or in a multiplexed format, particularly in planar and liquid array formats.

### BACKGROUND AND RELATED ART

Tissue homeostasis in normal and disease states is regulated by complex biological responses that are contributed by overlapping patterns of gene expression and protein activity. For this reason, disease states or specific biological responses usually cannot be characterized by a difference in the expression or activity of single genes or proteins, but by profiles that examine changes in the expression or activity of multiple genes or proteins simultaneously. For this reason, it is important to develop tools that can be used to measure the activity of many genes or proteins simultaneously.

Proteases are enzymes that cleave other proteins at specific peptide sequences. Caspases are a class of proteases that belong to a structurally related group of at least 14 different cysteine proteases. Caspase activities provide signatures for modes of apoptosis, the highly structured process of programmed cell death. Caspase-mediated proteolysis of cellular proteins is a critical event during apoptosis. The apoptotic response is critical for maintaining tissue homeostasis in normal and disease states and is an important target for therapeutic intervention. As crucial regulators of apoptosis, the caspase enzymes have been proven to have substantial clinical and pharmaceutical importance, and are currently widely studied in the pharmaceutical industry and in academic laboratories (1, 2, 3). Seven of these proteins, (caspase −2, −3, −6, −7, −8, −9, and −10), have central roles in mediating apoptosis and are divided into two classes based on function: the initiator caspases and the effector or executioner caspases. The initiator caspases including −2, −8, −9 and −10 are activated in response to cell death signals and activate one or more of the effector caspases (caspase −3, −6, and −7) that cleave specific cellular proteins to contribute to cell disassembly. In studying the effects of infection, disease, injury or pharmacologic agents on cells, it is important to determine which caspases are activated and where and when activation occurs, upon receipt of specific death stimuli. Such information will be useful in the design of strategies to regulate the activation of caspases during apoptosis.

Depending on the signal that initiates an apoptotic response and also on the presence of inhibitors specific for the individual caspases, different combinations of caspase activity contribute to apoptosis and provide signatures that may be associated with specific diseases or physiological and pharmacological agents (4, 5, 6). For example, caspase-8 is activated in response to the formation of a death-inducing surface signaling complex, whereas caspase-9 is triggered by cellular stress including DNA damage (7).

Assays capable of detecting and quantitating the activity of specific proteases in a sample are of substantial importance in biological and biochemical research, medicine, drug discovery, pharmacology, and diagnostics. Such assays can be used to elucidate biochemical pathways, identify pathological agents, to screen for potential therapeutic agents, to diagnose, classify and stage disease, and to determine the effectiveness of drug treatment. The effort to increase the utility and applicability of such assays is often frustrated by limitations of assay sensitivity, specificity and throughput. Understanding the complex biological processes involving proteases is also hampered by a lack of ability to measure the activity of multiple proteases simultaneously or near-simultaneously.

Methods for detecting and quantifying protease activity are commonly performed by mixing the protease with a labeled natural or synthetic protease substrate. The protease cleaves the substrate causing release of the label and thereby generating a measurable color or fluorescent signal (8, 9). However, studies on proteases in crude homogenates and subcellular fractions rather than purified proteases have been hampered by the lack of specific substrates to exclusively measure the activity of a specific proteases thus leading to expenditure of significant time and effort in the search for ever more specific substrates. Even if one finds a substrate that appears to be specific for a particular protease in a particular type of sample, there can be no assurance that different proteases will not be found in different sample types that will also cleave that substrate. For example, many of the proteases of the caspase family bind and cleave similar substrates, making it difficult to associate a particular protease activity with a specific caspase in vitro. Nevertheless, several different assays for caspase activity have been developed and commercialized (10). These assays utilizes a synthetic tetrapeptide, Asp-Glu-Val-Asp (DEVD), labeled with either a fluorescent moiety, 7-amino-4-trifluoromethyl coumarin (AFC), or a calorimetric label, p-nitroanilide (pNA) as substrates. DEVD-dependent protease activity is assessed by detection of the free AFC or pNA cleaved from the substrates. Different peptide sequences can be used to detect different caspase enzymes, but generally the caspases are not highly specific for these substrates, so that any particular peptide substrate may bind and be cleaved by multiple caspases simultaneously (11, 12).

It is apparent therefore, that improved methods and compositions for detecting and measuring protease activity, and in particular for measuring caspase activity, are greatly to be desired.

### SUMMARY OF THE INVENTION

It is therefore an object of the invention to provide new methods, systems and compositions for detecting and assaying protease activity and, in particular for detecting and assaying caspase activity.

In accordance with this object, there have been provided novel compositions and methods of the present invention that provide assays that are highly specific for individual proteases and enable simultaneous detection of multiple protease activities in a multiplex or array format. The present invention provides substantial improvement over the prior art in that one or more non-specific substrates or inhibitors (substrates that bind to the protease but cannot be cleaved) can be used to detect specific proteases and because the activities of multiple proteases can be assayed simultaneously or in a multiplex or array format. Furthermore, the present invention does not result in the release of a soluble signaling molecule so that detection of protease activity can be performed in a solid- or liquid-array format and thereby facilitates the use of signal amplification techniques that cannot be used when a soluble signal is generated.

In one embodiment, there is provided a detectable composition having a detectable complex immobilized upon a capture surface, where the detectable complex comprises a protease bound to a labeled inhibitor. The capture surface may comprise a specific recognition element, where the protease is immobilized upon the capture surface by binding to the specific recognition element. The specific recognition element may be, for example, an immunoglobulin such as a monoclonal or polyclonal antibody or antibody fragment, Protein G, Protein A, Protein A/G, a peptide, an oligonucleotide, nucleic acid, or a metal chelate. The capture surface may be, for example, a well, a substantially planar surface, or a particle, bead or microsphere, such as an individually addressable particle, bead or microsphere.

In another embodiment, there is provided a multiplex detection system, having a plurality of detectable compositions as described above. The system may have, for example, a substrate subdivided into a plurality of distinct loci, where each locus comprises a detectable complex immobilized on the surface of the locus, and where the detectable complex comprises a protease bound to a labeled inhibitor. The substrate may be, for example, a multiwell plate or a substantially planar surface, or may be an individually addressable particle, bead or microsphere. The particle, bead or microsphere may be magnetic and/or radio-frequency tagged.

In any of these compositions and/or systems, the labeled inhibitor may be labeled with a moiety selected from the group consisting of, for example, calorimetric labels, fluorescent labels, chemiluminescent labels, bioluminescent labels, biotin, digoxigenin, detectable carbohydrates, oligonucleotides, nucleic acids, peptides, polypeptides, protein, and glycoproteins. The labeled inhibitor may further comprise a binding moiety, for example, a fluoromethyl ketone, chloromethyl ketone, aldehyde, difluoromethyl ketone, diazomethyl ketone, OPH or DAP.

In another embodiment, there is provided a method of detecting a protease in a sample, including detecting the presence of a labeled complex on a capture surface, where the labeled complex is derived from the sample and comprises a protease bound to a labeled inhibitor. The detectable complex may be immobilized upon the capture surface by binding to a specific recognition element, where the specific recognition element binds to the protease. The specific recognition element, the capture surface, and the inhibitor may be as described above. The method may be multiplexed by detecting a plurality of labeled complexes compositions on a plurality of capture surfaces, where each labeled complex comprises a protease bound to a labeled inhibitor.

In any of these methods, the labeled complex may be prepared by contacting a sample suspected of containing a protease with a labeled inhibitor prior to, simultaneously with, or subsequent to, immobilization on the capture surface.

In any of these methods, systems, and compositions, the protease may be a caspase.

In any of these methods, systems and compositions, one or more reagents may be used to amplify the signal from the detectable label. The detectable label may comprise two or more labels such that the labels interact to produce a signal when they are in close proximity but fail to produce a signal when they are not in close proximity, or where the labels interact to a suppress a signal when they are in close proximity but produce a signal when they are not in close proximity.

In any multiplex system, an array may be used that comprises support and capture molecules such that more than one protease-inhibitor complex may be captured simultaneously or nearly simultaneously.

In another embodiment there is provided a kit for the detection, and/or quantitation of protease activity in a sample having one or more labeled inhibitors and an array having a support and one or more capture molecules. The kit may have additional components, for example, one or more purified proteases, one or more cell lysates or extracts, one or more quantitated standards or controls, one or more labeled secondary reagents, one or more reagents for amplification of the signal, one or more buffers for preparing the sample for analysis, one or more substances known believed to induce or inhibit protease activity in a sample and/or one or more unknown samples.

In another embodiment there is provided a method for measuring the binding of two or more inhibitors to one or more proteases in a sample having adding a first-labeled and second-labeled inhibitor to the sample, where the labels on the first-labeled and second-labeled inhibitors are different, incubating the mixture under conditions such that the inhibitors bind to the proteases in the sample, capturing the protease-inhibitor complexes onto an array having a support coated with capture molecules, and detecting the presence or absence and/or quantifying the amount of the first-labeled and second-labeled inhibitors bound to the support. Up to at least ten distinguishable labeled inhibitors may be used.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a schematic diagram of a Labeled Inhibitor containing one Binding Moiety and one Signal Moiety.

FIG. 2 shows a schematic diagram of a labeled substrate containing one binding moiety and one signaling moiety.

FIG. 3 shows a schematic diagram of a Labeled Substrate containing one binding moiety and two interacting signal moieties. The labels may interact to increase a signal or reduce a signal when they are in close proximity.

FIG. 4 shows a diagram of an array of capture molecules for use in the present invention. Each element of the array represents a region of the array that is bound with a capture molecule that is specific for a particular protease.

FIG. 5 shows a schematic diagram of a bead-based array. Each colored bead is bound with an antibody that is specific for a different protease. Two or more coated beads may be mixed to form an array.

FIG. 6 shows addition of the labeled inhibitor to the sample and binding of the inhibitor to the protease to form an inhibitor-protease complex.

FIG. 7 shows one way in which the inhibitor-protease complexes are captured onto a planar array and the signal at each element of the array can be measured with a microarray scanner.

FIG. 8 shows binding of protease-inhibitor complexes to a color-coded bead array. Identification of the bead color and measurement of the signal bound to each bead is performed in a flow cytometer.

FIG. 9 shows addition of a FRET-labeled substrate to a sample and binding to a protease forming a substrate-inhibitor complex. The protease subsequently cleaves the substrate between the two FRET labels and the quenching FRET label is no longer in close proximity of the fluorescent FRET label. Due to the binding moiety, the portion of the substrate containing the fluorescent FRET labels remains bound to the protease and now generates a fluorescent signal. This complex can now be captured onto an array.

FIG. 10 shows the median fluorescence units generated by different samples treated with and without the apoptosis inducer camtothecin and a biotin-labeled caspase 3 inhibitor.

FIG. 11 shows multiplex measurement of three different protease activities in a single reaction using an antibody-coated bead array to capture three different protease-inhibitor complexes.

FIG. 12 shows multiplex measurement of protease activities in a multiplex, competitive assay format. The activity of 3 caspase enzymes was measured in the presence or absence of the apoptosis inducer camptothecin and a labeled inhibitor. Another set of samples were run that included the experimental apoptosis inhibitor, MX435.

### DETAILED DESCRIPTION OF THE INVENTION

The present invention provides compositions and methods for assaying the presence and/or activities of proteases in a sample. In particular, the invention provides compositions and methods for simultaneously assaying multiple proteases in one or more samples.

Specifically, proteases are detected by immobilization on a capture surface prior to, simultaneously with, or subsequent to, binding to labeled inhibitor molecules. The inhibitor, as further defined below, is a moiety that binds to the protease in a manner that is sufficiently stable to permit detection of the protease-inhibitor complex. The protease may be bound to the inhibitor covalently or non-covalently. The inhibitor may or may not be cleaved by the protease, but when cleavage occurs a labeled portion of the inhibitor remains bound to the protease. The inhibitor may be directly labeled, for example with a dye, or may be labeled with an indirectly detectable moiety, for example, biotin. When the inhibitor is labeled with an indirectly detectable moiety, the inhibitor/protease complex may subsequently be detected with a labeled reagent that binds to that moiety. For example, when the inhibitor is labeled with biotin, the complex may subsequently be detected using streptavidin that is either directly labeled, for example with a dye, or that is conjugated to a molecule, such as an enzyme, that permits detection after a further chemical reaction, for example, with an enzyme substrate.

The capture surface comprises a capture molecule that specifically binds to either the protease or the protease-inhibitor complex. Suitable capture molecules include immunoglobulins, such as monoclonal or polyclonal antibodies and antibody fragments. Other suitable capture molecules are known in the art. In an embodiment where the protease is bound to the capture surface prior to binding with the inhibitor, the capture molecule binds to the protease in a manner that does not prevent inhibitor binding. In embodiments where the protease is bound to the capture surface after binding with the inhibitor, the capture molecule binds to the protease in a manner that does not disrupt the protease-inhibitor complex. In this case, binding may occur to the protease or to the protease-inhibitor complex.

The capture surface may be a continuous surface, for example a substantially planar surface, that can be subdivided, if desired, into discrete regions to permit the preparation of an array of a plurality of capture molecules. The regions may be of any physical form that is suitable for detection of the protease inhibitor complex and may include, without limitation, spots on a planar surface or wells on a plate. For multiplexed detection of a plurality of proteases, each of the discrete regions can contain a capture molecule that is specific for a particular protease or protease-inhibitor complex. The array may, if desired, contain multiple regions having the same capture molecule, permitting comparison between the regions for control purposes.

The capture surface may also be a particle, bead, microsphere or similar moiety. When the capture surface is particulate in this fashion, it is convenient to use individually addressable particulate structures that permit identification and separation of the structures. Individually addressable structures are known in the art and include magnetic beads, radio-frequency tagged particles, fluorescently labeled microspheres and the like. In such cases the presence of bound detection complex (comprising the inhibitor bound to the protease) can be detected by virtue of the presence of the label present on the inhibitor and the addressable moiety present on the structure. For example, a fluorescently labeled bead can be detected using flow cytometry, as discussed in more detail below.

In one aspect of the invention, a sample containing the protease(s) of interest is mixed with a labeled protease inhibitor that binds to the protease(s), but is not cleaved by the protease. The protease-inhibitor complexes are then captured onto a support that is coated with specific capture molecules such as mono- or polyclonal antibodies or antibody fragments. The capture molecule may specifically bind to the protease component of the protease-inhibitor complex, or it may specifically bind to the protease-inhibitor complex, that is, it may specifically bind only to one or more molecular structures that are present only in the complex. In this latter case, the capture molecule will not recognize either the uncomplexed protease or the uncomplexed inhibitor. The capture molecules can be bound to individually addressable beads or can be bound to a support in an array format. The signal from the bound substrate or inhibitor is then measured to quantify the protease binding activity.

In another aspect of the present invention, the sample containing the proteases is mixed with a labeled protease substrate that binds to the protease. The labeled substrate contains two labels that interact so that a signal is generated with one of the labels is cleaved by the action of the protease. Alternatively, the labels may interact such that they produce a signal when in close proximity, but fail to produce a signal when one of the labels is cleaved from the protease. The protease-substrate complexes are then captured onto a solid phase that is coated with specific capture molecules such as mono- or polyclonal antibodies or antibody fragments. The capture molecules can be bound to individually addressable beads or can be bound to a support in an array format. The signal from the bound substrate is then measured to quantify the protease activity.

In another aspect of the present invention, the signals from the labels on the captured protease-inhibitor or protease-substrate complexes are amplified by methods known in the art such as tyramide signal amplification or amplification by labeled oligonucleotide dendrimers (13, 14).

In another aspect of the present invention, the protease-substrate or protease-inhibitor complexes are first bound to the free capture molecules such as poly- or monoclonal antibodies and then these antibody-protease-inhibitor or antibody-protease-substrate complexes are subsequently captured onto the support.

Definitions and Abbreviations.

In the description that follows, a number of terms used in the field of biochemical assays in general and proteases in particular are utilized.

Protease

As used herein, “protease” refers to any peptide, polypeptide or peptide- or polypeptide-containing substance that catalyzes the hydrolysis of a protein or peptide. The protease may be natural or non-naturally occurring and may be isolated from a natural source, may be recombinant or synthetic and is not required to be in any particular form. Examples of well known proteases include bromelain, cathepsin B, cathepsin D, cathepsin G, chymotrypsin, clostripain, collagenase, dispase, endoproteinase Arg-C, endoproteinase Asp-N, endoproteinase Glu-C, endoproteinase lys-C, factor Xa, kallikrein, papain, pepsin, plasmin, proteinase K, subtilisin, thermolysin, thrombin, trypsin, acylamino-acid-releasing enzyme, aminopeptidase M, carboxypeptidase A, carboxypeptidase B, carboxypeptidase P, carboxypeptidase Y, cathepsin C, leucine aminopeptidase, and pyroglutamate aminopeptidase.

Sample

As used herein, “sample” refers to any composition or material that might contain a protease, and may include, without limitation, human and animal tissues, cultured cells, cultured or naturally occurring microorganisms, bodily fluids, blood, serum, and the like. The sample need not contain only the biological material. The sample may also consist of a protease-containing material on or in a physical matrix.

Lysis Reagent

As used herein a “lysis reagent” refers to any composition used to disrupt cells and tissues such that proteolytic enzymes are released and available for detection and/or capture. The composition may contain, without limitation, chemicals, salts, buffers and detergents that are commonly known and used in the art to prepare samples for analysis. The composition may also contain preservatives and stabilizers to stabilize and prevent degradation of the sample during storage, handling, and testing.

Inhibitor

As used herein, an “inhibitor” is a moiety that binds to the active site of a protease, but is not cleaved by the protease. The inhibitor may be labeled with one or more additional moieties such as signal moieties or a binding moieties.

Substrate

As used herein, a “substrate” is a molecule that binds to the active site of a protease and is cleaved by the protease. After cleavage, part of the substrate may remain bound to the protease.

Binding Moiety

As used herein, a “binding moiety” is a chemical moiety that is attached to a protease inhibitor or substrate that provides for increasing the binding affinity of the inhibitor to the protease. The binding moiety may form a non-covalent bond, a reversible covalent bond, or an irreversible covalent bond between the inhibitor or substrate and the protease. Examples of binding moieties are the aldehyde moiety (CHO), fluoromethylketone (FMK), and chloromethyl ketone (CMK). Protease substrates and inhibitors are commercially available that are labeled with a binding moiety.

Signal Moiety

As used herein, a “signal moiety” is a detectable label. In the present invention, a wide variety of signal moieties are possible including, for example, radioactive isotopes, fluorescent labels, chemiluminescent labels, bioluminescent labels, and enzyme labels. The signal moieties can also be haptens that are be recognized by secondary reagents such as antibodies, peptides, direct chemical interactions, and other methods that are well known in the art. The signal moiety may also be an oligonucleotide or nucleic acid that can be detected by hybridization, polymerization, ligation and/or amplification by methods well known in the art. The signal moiety may also comprise two chromophores bound in close proximity to utilize a phenomenon called fluorescence resonance energy transfer (FRET). When illuminated with light of the appropriate wavelength, one chromophore absorbs a photon and then exists in the excited state. The energy from the excited chromophore is transferred to an acceptor molecule when the two molecules are in close proximity to each other. This transfer prevents the excited chromophore from releasing the energy in the form of a photon of light thus quenching the fluorescence of the chromophore. When the acceptor molecule is not sufficiently close, the transfer does not occur and the excited chromophore may then fluoresce. Such pairs of interacting signal moieties are well known in the art (15, 16). A similar phenomenon known as luminescence resonance energy transfer (LRET) occurs between sensitized lanthanide metals and acceptor dyes and may be used in the present invention.

Capture Molecules

In the present invention, a “capture molecule” can be any molecule that will specifically capture a protease from a solution containing one or more biological molecules. Examples of capture molecules are poly- and monoclonal antibodies, synthetic, humanized or phage displayed antibodies and antibody fragments. The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). The term “synthetic antibody” as used herein, denotes an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. Proteins that have natural affinity for specific proteases and proteins that have been engineered to specifically bind to the protease are also included in the invention. Capture molecules may also be molecules that bind to another molecule that binds the protease. For example, anti-rabbit IgG may be used to capture a rabbit antibody-protease complex. Similarly, protein G may be used to capture a goat antibody-protease complex.

Support

As used herein, “support” may be any porous or non-porous material or matrix suitable for attaching capture molecules such as proteins, peptides, nucleic acids and the like. The capture molecules may be bound covalently or non-covalently to the support by any technique or combination of techniques well known in the art. Supports of the invention may comprise nylon, nitrocellulose, diazonitrocellulose, glass, silicon, polystyrene, polyvinyl chloride, polypropylene, polyethylene, dextran, sepharose, agar, starch, or any other material that allows for the immobilization of biomolecules. The material can be formed in filters, membranes, flat surfaces, tubes, channels, wells, sheets, beads, microspheres, columns, fibers (e.g. optical fibers) and the like. The support may also comprise multiwell tubes (such as microtiter plates) such as 12-well, 24-well 48-well, 96-well, 384-well, and 1537-well plates. Preferred beads are made of glass, latex, or a magnetic material (magnetic, paramagnetic, or supermagnetic beads). In the present invention, the support is preferably a set of color coded microspheres such as those manufactured and sold by Luminex Corporation (Austin, Tex.) (17).

Array

As used herein, the term “array” refers to an orderly arrangement of capture molecules on a support. Such arrays may be formed on micropiates, glass slides, beads, microspheres, microfluidic devices or standard blotting membranes and may be referred to as “arrays”, microarrays, or chips. Capture molecules may be bound to the support through covalent or non-covalent interactions. When bound to a planar surface, the capture molecules are bound in an orderly fashion such that the identity of any particular capture molecule can be identified by its position on the array. Such arrays may be constructed on planar objects such as glass or plastic microscope slides. Such arrays may also be constructed on the inside surface of a tube or microplate well or may be constructed inside the channels of a microfluidic device. In general, there is no restriction on the type of or format of the array as long as the individual sites to which the capture molecules are bound can be identified. If the support is a set of beads or microspheres, then sets of beads or microspheres coupled to different capture molecules must be distinguishable in some way. Beads from Luminex Corporation (Austin, Tex.) are color-coded by the addition of two different dyes at 10 different concentrations resulting in 100 different color beads. Capture molecules can be bound to specific bead colors and the color of each bead can be identified by flow cytometry. A bead array is prepared by binding specific capture molecules to sets of beads of a specific color, and then mixing different sets of colored beads to create an array. Similarly, microparticles from Pharmaseq (Princeton, N.J.) each contain a unique radio frequency tag that can be used to identify specific microparticles. Other methods can be used to tag individual beads for identification such as nucleic acid and peptide tags. The array may contain anywhere from 2 to 100,000 elements, preferably, between 3 and 5000 elements.

Signal Amplification

As used herein “signal amplification” refers to any method used to increase the signal of a biological assay beyond what can be achieved with a “one-label” detection strategy. Signal amplification may be based on an enzyme catalyzed reporter deposition such as tyramide signal amplification or may be based on enzyme amplification (18, 19). Alternatively, strategies that increase the number of labels may be used. Such strategies include the binding of dendrimers, branched polymers, and long linear polymers that contain multiple binding sites for a secondary detectable reagent. Examples of these strategies include oligonucleotide dendrimers, branched DNA, and Hybrid Capture (20, 21, 22). Nucleic acid amplification methods such as polymerase chain reaction and rolling circle amplification may also be used to amplify the signal obtained (23, 24). Although many of these strategies were designed to increase the sensitivity of detecting nucleic acids, they can be readily adapted to detection of other molecules simply by attaching an appropriate piece of nucleic acid to a detection reagent such as an antibody, peptide, avidin, or streptavidin. In the present invention, any method of signal amplification may be used to increase the signal generated by the assay.

General Procedure

Preparation of the Array

The array of the present invention is prepared by methods well known in the art of binding nucleic acids, peptides and proteins to supports (25, 26, 27). Capture molecules such as antibodies can be bound passively to glass or polystyrene that has been treated with gamma radiation. The capture molecules are held on the surface through strong hydrophobic interactions. Capture molecules may also be coupled to latex beads containing surface carboxylate functionality with the carbodiimide EDC and n-hydroxysuccinimide. For planar surfaces, the capture molecules can be spotted onto the surface with an automated spotting instrument such as those that are commonly used for preparing DNA microarrays (gene chips). For coupling to microspheres, coupling to capture molecules may be done in solution. After the capture molecules have been bound, the microspheres bound to different capture molecules are mixed to create the array.

Inhibitors and Substrate

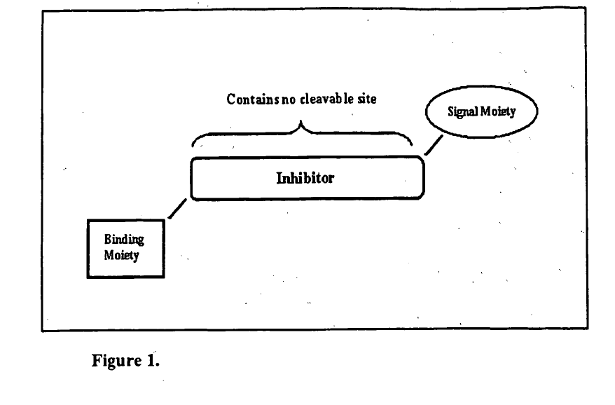

The preferred inhibitors of the present invention are peptides that bind strongly to proteases and contain a signal moiety. The necessity of using a binding moiety will be determined by the strength of the binding of the inhibitors or substrates to the protease. If the natural binding affinity is strong enough, then a binding moiety is not necessary. If the natural binding affinity is not strong, then it is necessary to have a binding moiety on the inhibitor or substrate. A schematic diagram of the preferred inhibitor is shown in FIG. 1. An example of a preferred inhibitor is biotin-DEVD-FMK. biotin-DEVD-FMK is an inhibitor that binds to the protease Caspase 3. FMK is a binding moiety that reacts covalently with the protease after binding. Biotin is the detection moiety that that can be detected with labeled avidin or streptavidin.

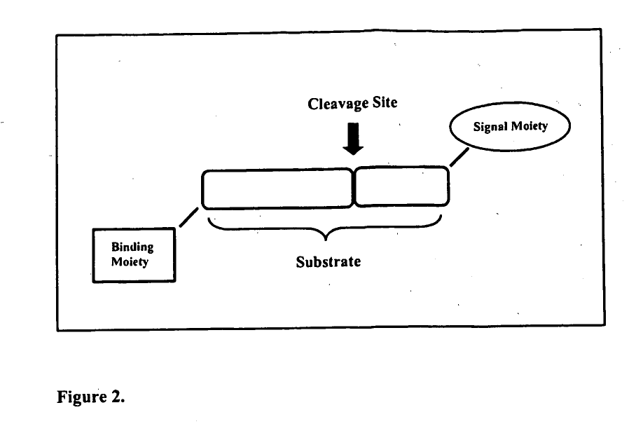

One type of preferred substrates of the present invention are peptides that bind covalently to proteases and contain a signal moiety. A schematic diagram of the preferred substrates is shown in FIG. 2.

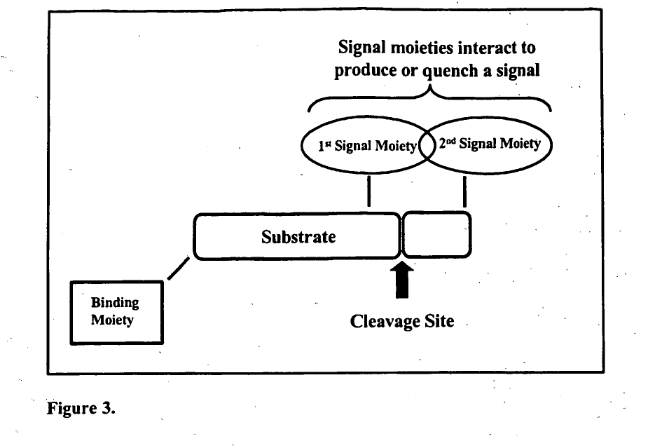

Another type of preferred substrates of the present invention are peptides that bind irreversibly to proteases and contain two interacting detection moieties. A schematic diagram of the these preferred substrates is shown in FIG. 3.

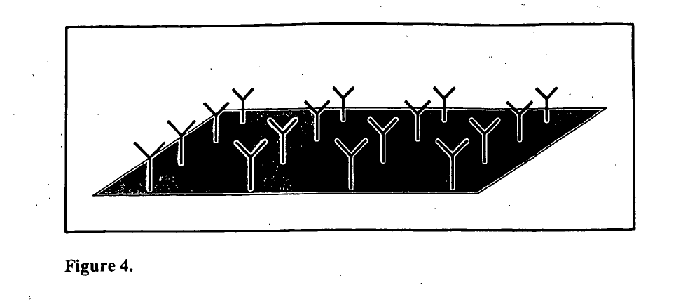

The preferred capture molecules of the present invention are poly- or monoclonal antibodies that are specific for particular proteases and do not cross-react with related proteases. Such antibodies are commercially available from suppliers such as Santa Cruz Biotechnology (Santa Cruz, Calif.). The antibodies may be bound to a planar support to create an array of capture molecules as shown in FIG. 4.

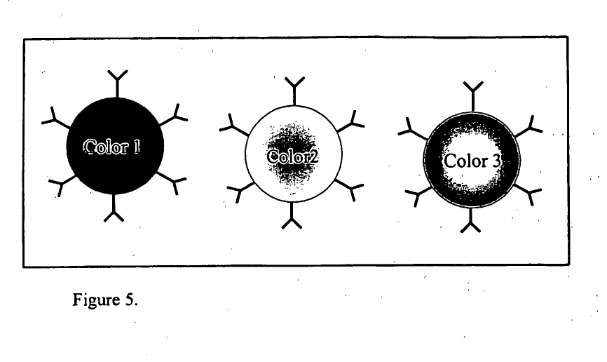

The preferred support of the present invention are sets of color coded microspheres such as those produced and sold by Luminex Corporation (Austin, Tex.). Antibodies can be bound to the microspheres using the carbodiimide EDC and n-hydroxysuccinimide. A diagram of a bead array is shown in FIG. 5.

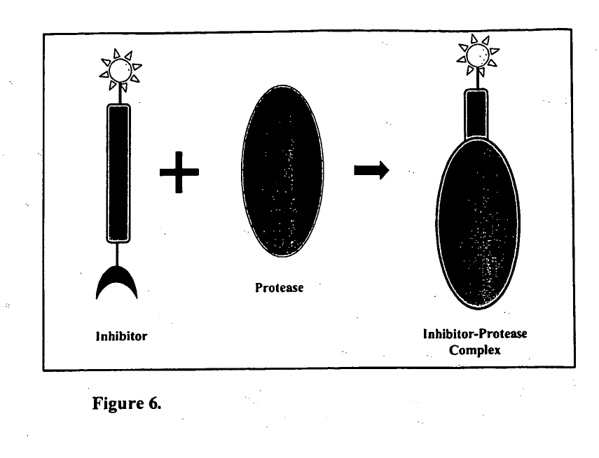

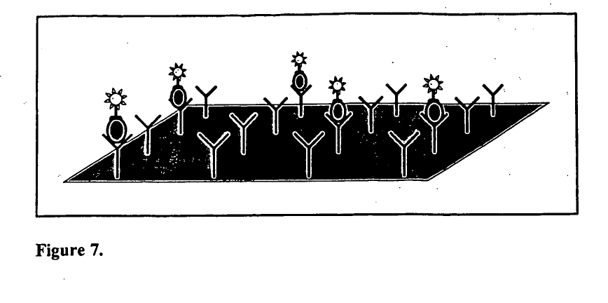

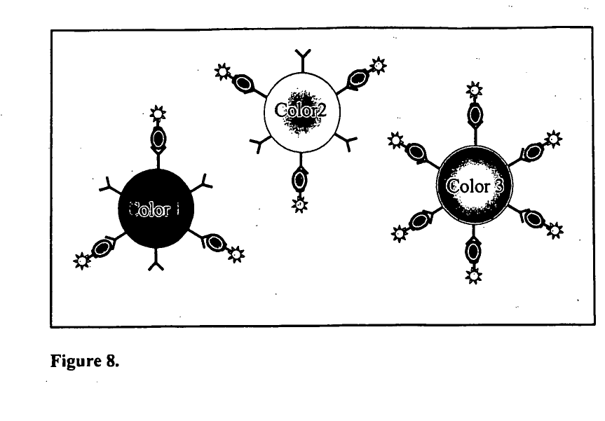

In the preferred mode of the present invention, a labeled inhibitor is added to one or more cell cultures. Typically, the inhibitor will be added to one control culture and one or more test cultures that are being grown in conditions that are different than those grown in the control culture. The inhibitor is taken up by the cells and binds to the active proteases in the cells. The cells are then lysed with a lysis reagent and the cell lysate is contacted with the array. Protease-inhibitor complexes are captured by antibodies on the array, preferably in a fashion that keeps the lysate moving with respect to the element of the array. These steps are illustrated in FIGS. 6-8.

Static capture in which the lysate is not in motion relative to the array is also possible, although it will take much longer to capture the same amount of protease-inhibitor complex. If the array is comprised of a set of color-coded beads, then the bead array and all or a portion of the cell lysate may be mixed during the capture step to ensure the most efficient capture of the protease-inhibitor complexes. Once the capture is complete, the excess cell lysate is washed from the array. In the case of the bead array, the washing may be accomplished by either centrifugation or filtration. After washing, a planar array may be scanned using a scanner commonly used for gene chip analysis. For the color-coded bead array, the array is read by a 2 or 3-channel flow cytometer. One or two channels determine the color of each bead thereby determining the identity of the capture molecule bound to that bead. The remaining channel is used to measure the fluorescent intensity of the label attached to the inhibitor on each bead. Once a full array or a full set of beads has been read, a protease activity profile can be generated for each sample by measuring and recording the activity of each protease in each sample. In this mode of the present invention, the inhibitor may be labeled directly with a fluorescent label or with a hapten such as a peptide, biotin or digoxigenin and the detection may be accomplished through the use a labeled secondary reagent such as a labeled antibody, streptavidin-phycoerythrin or antidigoxigenin-fluorescein.

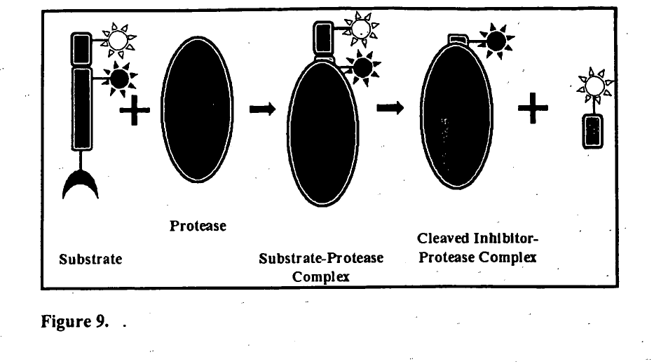

In another aspect of the present invention, a FRET-labeled substrate is added to one or more cell cultures. Typically, the substrate will be added to one control culture and one or more test cultures that are being grown in conditions that are different than those grown in the control culture. The substrate is taken up by the cells and binds to the active proteases in the cells. The substrate is then cleaved resulting in the creation of a fluorescent signal bound to the remaining part of the substrate that is bound to the protease as illustrated in FIG. 9. The cells are then lysed with the lysis reagent and the cell lysate is contacted with the array. Protease-substrate complexes are captured by antibodies on the array, preferably in a fashion that keeps the lysate moving with respect to the element of the array. Static capture in which the lysate is not in motion relative to the array is also possible, although it will take much longer to capture the same amount of protease-substrate complex. If the array is comprised of a set of color-coded beads, then the bead array and all or a portion of the cell lysate may be mixed during the capture step to ensure the most efficient capture of the protease-substrate complexes. Once the capture is complete, the excess cell lysate is washed from the array. In the case of the bead array, the washing may be accomplished by either centrifugation or filtration. After washing, a planar array may be scanned using a scanner commonly used for gene chip analysis. For the color-coded bead array, the array is read by a 2 or 3-channel flow cytometer. One or two channels determine the color of each bead thereby determining the identity of the capture molecule bound to that bead. The remaining channel is used to measure the fluorescent intensity of the label attached to the substrate on each bead. Once a full array or a full set of beads has been read, a protease activity profile can be generated for each sample by measuring and recording the activity of each protease in each sample.

In another aspect of the invention a labeled inhibitor and a non-labeled inhibitor are both added to the sample. The non-labeled inhibitor competes with the labeled inhibitor for binding to proteases. With this mode of the invention, new inhibitors may be screened for specificity and the binding constants of new inhibitors may be determined by determining the amount of unlabeled inhibitor necessary to reduce the binding of the labeled inhibitor by a certain amount.

In another aspect of the invention, a first-labeled inhibitor and a second-labeled inhibitor are both added to the sample wherein the labels on the two inhibitors are different and may be detected independently. The first- and second-labeled inhibitors may compete for binding to the same protease or may bind to different proteases or a combination of the two. The labels are then detected in a manner that allows the measurement of each label independently. For example, if the array is a glass slide on which one or more regions of the glass slide have been spotted with specific anti-protease antibodies, and the first inhibitor is labeled with the fluorescent dye Cy3 and the second-labeled inhibitor has been labeled with Cy5, then both inhibitors may be added to a sample, allowed to incubate and bind specifically to proteases. After incubation, the protease-inhibitor complexes in the sample are captured onto the array, the array is washed to remove non-bound sample and inhibitors. The array is then scanned with a dual-color fluorescent scanner such as are commonly used in DNA microarray analysis. In this mode of the invention, multiple inhibitors may be screened against multiple of proteases simultaneously, the only limitation being the number of different labels that may be detected. At the current state of fluorescent scanning technology, up to 4 labels can now be scanned in a single instrument.

In another aspect of the invention, some of the samples are treated with a known activator of protease activity, and experimental compounds designed to inhibit protease activity. This mode of the invention may be used to screen compound libraries to find compounds that may inhibit protease activity.

In another aspect of the present invention, the protease-inhibitor or protein-substrate complexes are captured onto the array and the detection moiety is detected with one or more reagents that amplifies the signal. For example, if the detection moiety is biotin, the biotin may be detected with a streptavidin-peroxidase conjugate. Subsequently, the peroxidase can be detected by tyramide signal amplification (18). Alternatively, the streptavidin used to detect the biotin may be labeled with a long RNA:DNA hybrid or a single strand or DNA to which a strand of RNA can be subsequently bound. The RNA:DNA hybrid can then be detected with a fluorescent-labeled anti-RNA:DNA antibody. Since multiple antibodies can be bound to a single RNA:DNA hybrid, the signal is significantly greater than if the streptavidin was labeled with a fluorescent dye. It will be readily apparent to one of ordinary skill in the art that any method of signal amplification can be adapted for use in conjunction with the present invention.

The present invention also relates to kits for the detection and measurement of protease activity in one or more samples, particularly for the creation of protease activity profiles wherein, the activity of two or more proteases are measured in a multiplex or array format. Such kits may be diagnostics kits wherein the presence or absence or level of a protease or multiple proteases is correlated with the presence or absence of a disease or disorder. The invention also relates to kits for making the composition of the invention.

In specific embodiments, the kits comprise an array, and one or more protease substrates or inhibitors. The kit can further comprise additional components for carrying out the detection/quantitation assays of the invention. Such kits may comprise one or more additional components selected from the group consisting of one or more purified proteases, one or more cell lysates or extracts, one or more quantitated standards or controls, one or more labeled secondary reagents, one or more reagents for amplification of the signal, one or more buffers for preparing the sample for analysis, one or more reagents for preparing the sample for analysis, one or more chemicals to induce or inhibit protease activity in a sample and one or more unknown samples. The kits of the invention preferably comprise a container (box, carton, or other packaging, having in close confinement therein one and preferable more containers, (tubes, vials, and the like) which comprise various reagents for carrying out the method of the invention. The reagents may be in separate containers or may be combined in different combinations in a single container. Such kits of the invention may further comprise instructions or protocols for carrying out the methods of the invention and optionally, may comprise an apparatus or other equipment for detecting the detectable labels associated with the inhibitors or substrates of the invention. The kit may include a computer program or internet access to a computer program for data acquisition and analysis of the protease activity and generation and analysis of protease activity profiles.

It will be readily apparent to one of ordinary skill in the relevant arts that other suitable modifications and adaptations to the methods and applications described herein may be made without departing from the scope of the invention or any embodiment thereof. Having now described the invention in detail, the same will be more clearly understood by reference to the following examples which are included herewith for purposes of illustration only and are not intended to be limiting of the invention. Thus, the invention should in no way be construed as being limited to the following examples, but rather, should be construed to encompass any and all variations which become evident as a result of the teaching provided herein.

As used herein and in the following claims, articles such as “the”, “a” and “an” can connote the singular or plural.

All documents referred to herein are specifically incorporated by reference in their entireties.

The present invention, thus generally described, will be understood more readily by reference to the following examples, which are provided by way of illustration and are not intended to be limiting of the present invention

### EXAMPLE 1

### General Example of an Array to Detect Protease Activity

Antibodies or other molecules such as but not limited to peptides, polypeptides, native or engineered proteins, nucleic acids, carbohydrates, and lectins that bind to one or more proteases are coupled to a support through amine, thiol, carboxylic acid, carbohydrate groups using standard coupling chemistries or through non-covalent binding. The assays make use of detection schemes that utilize inhibitors or substrates of proteases that are labeled with UV or visible dyes or fluorophores, or that contain tags that present detectable labels such as biotin molecules, chemicals, peptides, nucleic acids or carbohydrates that are used in conjunction with labeled antibodies, peptides, forms of avidin, chemicals, nucleic acids, peptides, or proteins. The substrates or enzyme inhibitors may have specificity to one or more proteases within or across classes of enzymes and will bind active enzymes. Cell or tissue extracts, cells or tissues in culture or permeabilized cells and tissues of eukaryotic or prokaryotic origin are treated with inhibitors or substrates of proteases to be used as detection molecules. Extracts from cells or tissues or permeabilized cells or tissues labeled with protease substrates or inhibitors are incubated with supports coupled to molecules that have binding specificity for one or more proteases. Proteases captured on support surfaces are quantified by assaying for the protease bound detection molecules. Assays may be conducted in homogenous, heterogeneous, and other formats.

### EXAMPLE 2

### A Single-Plex Assay For Active Caspase-3

Preparation of Amine-Reactive Beads

Luminex beads were dispersed by vortexing for 15 seconds followed by sonication in a water bath for 2 minutes. To wash, beads were pelleted by centrifugation for 2 minutes at 12,000×g in a microcentrifuge and then resuspended in 100 ul of water. Beads were pelleted again, resuspended in 80 ul of activation buffer (100 mM sodium phosphate, monobasic pH 6.3), vortexed briefly and sonicated for 30 seconds. Reactive n-hydroxysuccinimide (NHS) esters on the bead surface were prepared by adding 10 ul of 50 mg/ml Sulfo-NHS solution (Pierce, Rockland, Ill.) and 10 ul of 50 mg/ml 1-Ethyl-3-[3-dimethylaminopropyl]carbodiimide Hydrochloride (EDC) to the bead suspension, briefly vortexing, and rotating for 20 minutes in the dark. The NHS-modified beads were washed twice with 100 ul of 50 mM MES buffer (pH 5.0-6.0) and then resuspended in 150 ul MES buffer (pH 5.0-6.0).

Coupling of Antibodies to Bead Surfaces

Five micrograms of purified antibody specific for caspase-3 (Santa-Cruz Biotechnology Inc.) provided in PBS buffer pH 7.4 was brought to 350 ul volume with MES buffer pH 5.5 and then added to the 150 ul NHS-bead suspension. The antibody-bead suspension was reacted at room temperature for 2 hours in the dark while mixing during which amine groups on the antibody reacted with the NHS esters on the bead surface forming a stable amide bond. Following the coupling reaction, beads were washed twice with 250 ul PBS-TBN (phosphate buffered saline with 0.1% BSA 0.02% tween-20) resuspended in a final volume of 200 ul PBS-TBN, and stored at 4 degrees.

Generation Of Lysates Containing Labeled Caspase Enzymes.

Human myeloma cell line KAS was cultured in RPMI+10% FBS at 37° C. in the presence of 5% CO₂. Cells in log phase growth were treated for 8 hours with 5 uM camptothecin, a known inducer of apoptosis, in the presence or absence of 1 uM biotinylated caspase inhibitor. A control culture was also prepared that contained the labeled inhibitor, but was not treated with camptothecin. The caspase inhibitor, biotin-DEVD-FMK (Enzyme Systems Products) binds irreversibly to caspase 1, 2, 3, 6, 7, and 9. Cells were then washed by centrifugation at 700×g for 5 minutes in a table-top centrifuge and were resuspended in cold PBS pH 7.4. After three washes, the cell pellet was vortexed in lysis buffer containing 50 mM Tris-HCl pH 7.5, 150 mM NaCl, 1 mM EDTA, 0.5% NP40, 0.5% Triton X-100 and protease inhibitors (5 ug/ml leupeptin, 2 ug/ml pepstatin A, 1 mM PMSF (phenyl methyl sulfonyl chloride). Lysates were stored overnight at −80° C., thawed on ice, vortexed, and spun at 4° C. for 1 hour at 12,000×g in a micro-centrifuge. Protein concentrations in the cleared supernatants were determined spectrophotometrically using Bradford reagent prior to storage at −80° C.

Capture Of Active Caspase-3-Inhibitor Complex On Antibody-Coupled Luminex Beads

Control beads and beads coupled to anti-caspase 3 antibodies were vortexed for 30 seconds and sonicated for 2 minutes in a water-bath sonicator to disperse the beads. Lysates from cells treated with camptothecin alone, camptothecin and biotinylated caspase inhibitor, or biotinylated caspase inhibitor alone were diluted in assay buffer (50 mM Tris pH 7.5, 100 mM NaCl, 0.05% tween-20 and 0.1% BSA) to obtain a final protein concentration of 10 ug/ml. 2 ul of the bead suspension (1000 beads/ul) was added to 50 ul of each sample and incubated for 1 hour in the dark with gentle shaking. Beads were then washed with 100 ul of assay buffer using a filterplate comprising a 1.2 micron PVDF membrane (Millipore). Beads were resuspended in 50 ul of assay buffer containing 2 ug/ml streptavidin-PE conjugate (Pierce) and shaken in the dark for an additional 30 minutes. Following the binding of the PE-conjugate, beads were washed with 100 ul of assay buffer and resuspended in 50 mM Tris pH 7.5, 100 mM NaCl, 0.02% tween-20. The presence of biotinylated inhibitor bound to caspase 3 on the bead surface was detected using the Luminex 100 instrument.

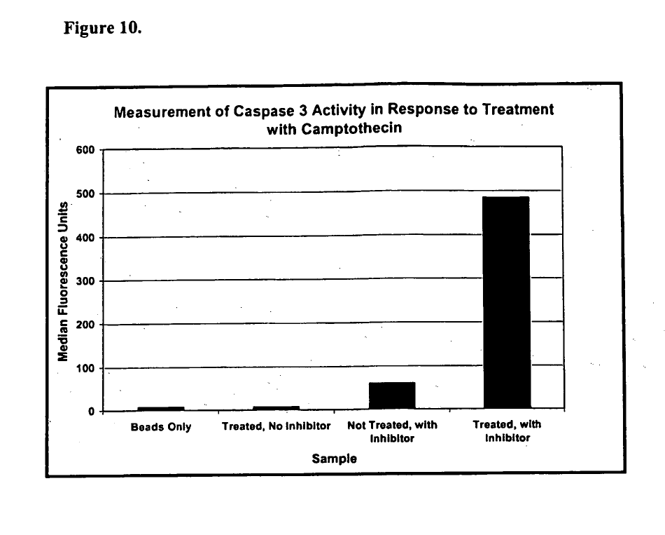

The signal detected on the beads is shown in FIG. 10. The sample treated with camptothecin but no inhibitor, gave signal equivalent to the beads only background as expected since no biotin-labeled inhibitor was bound to the caspase that was captured onto the beads. The sample that did contain inhibitor but that was not treated with camptothecin gave a low signal that reflects the low level of basal caspase activity in the cells. The sample that was treated with camptothecin and included biotin-labeled inhibitor gave a high signal thereby demonstrating that caspase 3 was induced by treatment with camptothecin.

### EXAMPLE 3

### Multiplex Caspase Assay

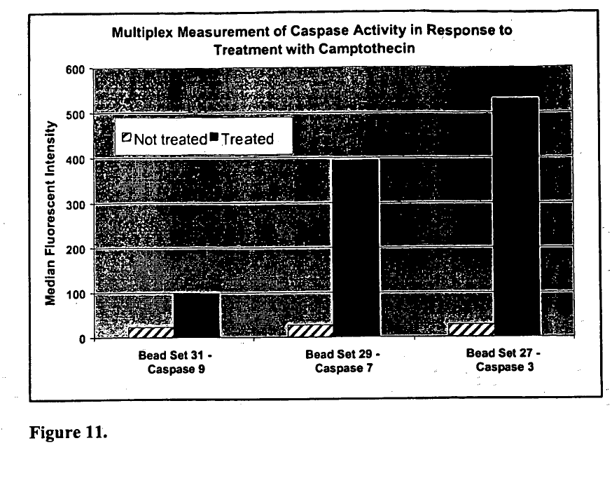

Using the method described in example 1 above Luminex bead sets (colors) 27, 29, and 31 are coupled to antibodies for caspase-3, caspase-7, and caspase 9 respectively. Immediately before using, beads are vortexed for 30 seconds and sonicated for 2 minutes to disperse. Beads are then mixed to provide a 1000 bead/ul (each color) suspension with equivalent representation of each bead set. Cell lysate was prepared from myeloma cell line Kas as describe in example 1. One culture was treated with biotinylated caspase inhibitor biotin-DEVD-FMK for 8 hours and a second culture was treated for 8 hours with the apoptosis inducer camptothecin and biotinylated caspase inhibitor, biotin-DEVD-FMK. A portion of the multiplex bead suspension (2 ul) was incubated with 50 ul of each sample of cell extract diluted to 10 ug/ml in assay buffer (50 mM Tris pH 7.5, 100 mM NaCl, 0.05% tween-20 and 0.1% BSA). Following a 1 hour incubation while shaking in the dark, beads are washed with 100 ul of assay buffer using a filterplate comprising a 1.2 micron PVDF membrane (Millipore). Beads are resuspended in 50 ul of assay buffer containing 2 ug/ml streptavidin-PE conjugate (Pierce) and shaken in the dark for an additional 30 minutes. Following the binding of the PE-conjugate, beads are washed with 100 ul of assay buffer and resuspended in 50 mM Tris pH 7.5, 100 mM NaCl, 0.02% tween-20. The presence of labeled caspase enzymes on bead surfaces was detected using the Luminex 100 instrumentation. A total of 100 events was collected for each bead set and the median fluorescent intensity was plotted in FIG. 11. As shown in FIG. 11, all three caspases are induced, but the level of induction was different for each specific caspase. Caspase 9 was induced the least while caspase 3 was induced the most.

### EXAMPLE 4

### Competitive Multiplex Inhibitor Assay

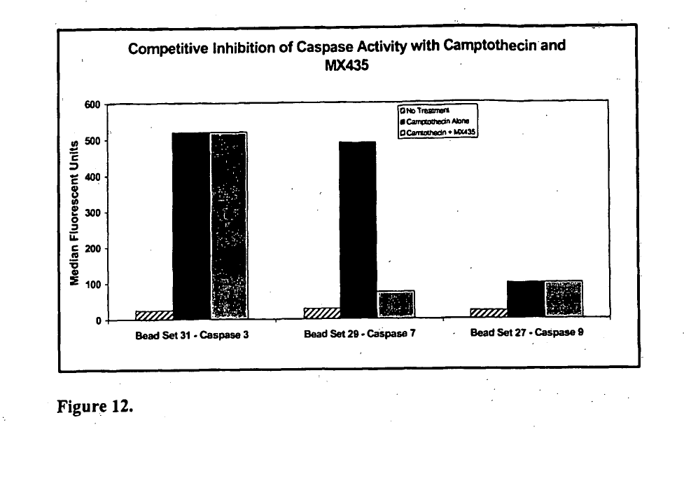

Using the methods described in example 2 above Luminex bead sets (colors) 27, 29, and 31 are coupled to antibodies for caspase-3, caspase-7, and caspase 9 respectively. Immediately before using, beads are vortexed for 30 seconds and sonicated for 2 minutes to disperse. Beads are then mixed to provide a 1000 bead/ul (each color) suspension with equivalent representation of each bead set. Cell lysate is prepared from the myeloma cell line Kas as described in Example 1 with the exception that an additional portion of cells is treated for 8 hours with camptothecin, the biotinylated caspase inhibitor biotin-DEVD-FMK and an equal amount of an experimental caspase inhibitor called Mx435. The MX435 inhibitor is not labeled with biotin, and therefore, it competes with the labeled inhibitor for binding to each caspase. A portion of the multiplex bead suspension (2 μl) is incubated with 50 ul of each sample of cell extract diluted to 10 ug/ml in assay buffer (50 mM Tris pH 7.5, 100 mM NaCl, 0.05% tween-20 and 0.1% BSA). Following a 1 hour incubation while shaking in the dark, beads are washed with 100 ul of assay buffer using a filterplate comprising a 1.2 micron PVDF membrane (Millipore). Beads are resuspended in 50 ul of assay buffer containing 2 ug/ml streptavidin-PE conjugate (Pierce) and shaken in the dark for an additional 30 minutes. Following the binding of the PE-conjugate, beads are washed with 100 ul of assay buffer and resuspended in 50 mM Tris pH 7.5, 100 mM NaCl, 0.02% tween-20. The presence of labeled caspase enzymes on bead surfaces is detected using the Luminex 100 instrumentation. A total of 100 events is collected for each bead set and the median fluorescent intensity is plotted in FIG. 12. As shown in FIG. 12, all three caspases are induced by the additional of camptothecin to the culture. In the presence of the experimental caspase inhibitor MX435, however, the induction of caspase 3 and caspase 9 activity is unchanged, but the activity or caspase 7 is greatly reduced. This result demonstrates that MX435 binds specifically to caspase 7 but not to caspase 3 or 9.

### REFERENCES

1. Gastman, BR. Head Neck. (2001) 23(5):409425.
2. Cohen G M. Biochem J. (1997) 326: 1-16.
3. Agress T. The Scientist (2001) 15(13):18.
4. Bratton, S B. et al., Adv. Exp. Med. Biol. (2001) 500:407-420.
5. Sartorious et al., Chembiochem (2001) 8;2(1):20-29.
6 Leung-Toung et al., Curr. Med. Chem. (2002) 9(9):979-1002.
7. Putcha et al., J. Cell Biol. (2002) 157(3):441453.
8. Brock et al., Clin Chim Acta (1978) 85(1):99-100.
9. Rinderknecht et al., (1975) 59(2):139-46.
10. GurtuetaL, (1997) 251(1):98-102.
11. Gorman et al., J Immunol Methods 1999 Jun. 24;226(1-2):43-8
12. Margolin et al., J Biol Chem (1997). 272(11):7223-8.
13. Heiskanen et al., Cancer Res (2000) 60(4):799-802.
14. Yu et al.,. Mol Vis (2002) 8:130-7.
15. Tyagi et al., Nat Biotechnol (1998) 16(1):49-53.
16. Kricka, Ann Clin Biochem (2002) 39(Pt 2):114-29
17. U.S. Pat. No. 5,981,180.
18. Hunyady et al., J Histochem Cytochem. (1996) 44(12):1353-62.
19. Johannsson et al., Clin Chim Acta (1985) 148(2):119-24.
20. Nilsen et al., Theor Biol (1997)187(2):273-84.
21. Urdea et al., Clin Chem. (1989) 35(8):1571-5.
22. Lazar et al., J. Clin. Ligand Assay 1999;22(2):139-151.
23. Gusev et al., Am J Pathol. (2001) 159(1):63-9.
24. Joerger et al., Clin Chem. (1995) 41(9):1371-7.
25. Schena et al., Proc Natl Acad Sci USA 1996. 93(20): 10614-9.
26. Wegner et al., Anal Chem. 2002. 74(20):5161-8.
27. Angenendt et al., Anal Biochem. 2002. 309(2):253-60.

1. A detectable composition comprising a detectable complex immobilized upon a capture surface, wherein said detectable complex comprises a protease bound to a labeled inhibitor.

2. The composition according to claim 1, wherein said capture surface comprises a specific recognition element, wherein said protease is immobilized upon said capture surface by binding to said specific recognition element.

3. The composition according to claim 2, wherein said specific recognition element is selected from the group consisting of an immunoglobulin, Protein G, Protein A, Protein A/G, a peptide, an oligonucleotide, nucleic acid, and a metal chelate.

4. The composition according to claim 3, wherein said specific recognition element is an immunoglobulin selected from the group consisting of monoclonal antibodies and antibody fragments, and polyclonal antibodies and antibody fragments.

5. The composition according to claim 1, wherein said capture surface is a well, a substantially planar surface, or a particle, bead or microsphere.

6. The composition according to claim 5, wherein said capture surface is an individually addressable particle, bead or microsphere.

7. A multiplex detection system, comprising a plurality of detectable compositions according to claim 1.

8. A multiplex detection system, comprising a plurality of detectable compositions according to claim 5.

9. A multiplex detection system, comprising a substrate subdivided into a plurality of distinct loci, wherein each locus comprises a detectable complex immobilized on the surface of said locus, and wherein said detectable complex comprises a protease bound to a labeled inhibitor.

10. The system according to claim 9, wherein said substrate is a multiwell plate or a substantially planar surface.

11. The system according to claim 9, wherein said substrate is an individually addressable particle, bead or microsphere.

12. The system according to claim 11, wherein said particle, bead or microsphere is magnetic and/or is radio-frequency tagged.

13. The composition according to claim 1, wherein said labeled inhibitor is labeled with a moiety selected from the group consisting of colorimetric labels, fluorescent labels, chemiluminescent labels, bioluminescent labels, biotin, digoxigenin, detectable carbohydrates, oligonucleotides, nucleic acids, peptides, polypeptides, protein, and glycoproteins.

14. The system according to 7, wherein said labeled inhibitor is labeled with a moiety selected from the group consisting of colorimetric labels, fluorescent labels, chemiluminescent labels, bioluminescent labels, biotin, digoxigenin, detectable carbohydrates, oligonucleotides, nucleic acids, peptides, polypeptides, protein, and glycoproteins.

15. A composition according to claim 1, wherein said labeled inhibitor further comprises a binding moiety.

16. A composition according to claim 15, wherein said binding moiety is selected from the group consisting of fluoromethyl ketone, chloromethyl ketone, aldehyde, difluoromethyl ketone, diazomethyl ketone, OPH and DAP.

17. A method of detecting a protease in a sample, comprising detecting the presence of a labeled complex on a capture surface, wherein said labeled complex is derived from said sample and comprises a protease bound to a labeled inhibitor.

18. The method according to claim 17, wherein said detectable complex is immobilized upon said capture surface by binding to a specific recognition element, wherein said specific recognition element binds to said protease.

19. The method according to claim 18, wherein said specific recognition element is selected from the group consisting of an immunoglobulin, Protein G, Protein A, Protein A/G, a peptide, an oligonucleotide, nucleic acid, and a metal chelate.

20. The method according to claim 19, wherein said said specific recognition element is an immunoglobulin selected from the group consisting of monoclonal antibodies and antibody fragments, and polyclonal antibodies and antibody fragments.

21. The method according to claim 17, wherein said capture surface is a well, a substantially planar surface, or a particle, bead or microsphere.

22. The method according to claim 21, wherein said capture surface is an individually addressable particle, bead or microsphere.

23. A method of detecting a plurality of proteases, comprising detecting a plurality of labeled complexes compositions on a plurality of capture surface, wherein each labeled complex comprises a protease bound to a labeled inhibitor.

24. A method according to claim 23, wherein each of said labeled complexes is arrayed on a distinct area of a multiwell plate or a substantially planar surface.

25. The method according to claim 23, wherein each of said capture surfaces is an individually addressable particle, bead or microsphere.

26. The method according to claim 25, wherein said particle, bead or microsphere is magnetic and/or is radio-frequency tagged.

27. The method according to claim 21, wherein said labeled inhibitor is labeled with a moiety selected from the group consisting of colorimetric labels, fluorescent labels, chemiluminescent labels, bioluminescent labels, biotin, digoxigenin, detectable carbohydrates, oligonucleotides, nucleic acids, peptides, polypeptides, protein, and glycoproteins.

28. The method according to claim 21, wherein said labeled complex is prepared by contacting a sample suspected of containing a protease with a labeled inhibitor prior to immobilization on said capture surface.

29. The method according to claim 21, wherein said labeled complex is prepared by contacting a sample suspected of containing a protease with a capture surface, followed by contacting the capture surface with a labeled inhibitor.

30. A composition according to claim 1, wherein said protease is a caspase.
